(19) United States
(12) Patent Application Publication
(10) Pub. No.: US 2005/0171468 A1
(43) Pub. Date: Aug. 4, 2005

(54) Gastric aspirate intestinal feeding tube

(76) Inventor: Scott Douglas Wood, Dallas, TX (US)

Correspondence Address: AKIN, GUMP, STRAUSS, HAUER & FELD, 1111 LOUISIANA STREET, 44TH FLOOR, HOUSTON, TX, 77002, US

(21) Appl. No.: 10/771,912

(22) Filed: Feb. 4, 2004

(51) Int. Cl. A61M 3/02

(52) U.S. Cl. 604/39

(57) ABSTRACT

A medical intubation device for delivering tube feeds into the duodenum or other intestinal area while aspirating the stomach of a patient is inserted through the patient's oral-nasal cavity. A first lumen of a tubular assembly allows delivery of fluids through outlets into the intestines. A second lumen of the tubular assembly interior to the first lumen provides aspiration through outlets into the stomach. A third lumen of the tubular assembly interior to the second lumen provides ventilation of the second lumen. A weighted member can be coupled at the insertion end of the tubular assembly. A radio-opaque allows positioning of the outlets into the stomach.

### BACKGROUND OF THE INVENTION

1. Field of the Invention

This invention pertains to medical intubation devices for temporary insertion of a tube into a patient. More particularly, this invention relates to a multi-lumen flexible gastrointestinal tube allowing simultaneous intestinal feeding and gastric aspiration of the patient.

2. Description of the Related Art

Current clinical practice uses two main types of temporary feeding tubes for medical intubation. The first tube, often referred to as a G-tube or sump tube, is placed through the nares or the mouth with the distal end being positioned in the stomach. The tube is used to instill liquid feeds, fluids, and liquid medications into the stomach. Using this tube, the stomach can also be emptied by aspirating the gastric contents, by connecting the proximal end of the tube to a wall suction or applying negative pressure with a syringe. However, feeding and aspiration cannot be done simultaneously.

The ability to check residual volume and empty the stomach when needed is of significant value when treating critically ill patients. These patients often undergo procedures requiring them to be placed in a supine position, which generally cannot safely be done without first emptying the gastric contents to prevent unwanted aspiration. Patients who are critically ill often develop gastro paresis, in which gut motility is significantly reduced and may cease altogether. Therefore, when tube feeds are instituted, infusing should begin slowly and increased to goal rates only after documenting a lack of significant residual fluids. If high residuals remain unchecked, unwanted aspiration of feeds and medication into the lungs may occur, which could result in pneumonia. Aspiration pneumonia and its associated complications are major contributors of morbidity and mortality in such patients.

Thus, the ability to analyze not only the quantity but also the quality of gastric secretions can improve patient care. For example, when patients develop upper gastrointestinal bleeds, often the first indication is a change in the color and consistency of the gastric aspirate. Detecting the color change allows for a more timely intervention. Typically, a G-tube is made of a somewhat hard or firm plastic in order to avoid collapse and occlusion of the lumen when applying negative pressure for aspiration. Although some G-tubes have added a vent port within the main tube to attempt to prevent mucosal damage during continuous or intermittent suction, because the tube is firm, it can often irritate the nasal and oral mucosa, resulting in patient discomfort. G-tube use may cause a significant increase in the incidence of acute sinusitis, nasal ulceration, and other associated complications.

Because the distal end of a G-tube is in the stomach, impaired or delayed gastric emptying can be a major limiting factor in the amount of feeds that can be provided. Duodenal feeding uses a feeding port that is distal to and therefore bypasses the pyloric sphincter. A second known type of feeding tube, one example of which is known as a Dobbhoff tube, allows feeding past the pyloric sphincter into the duodenum. Duodenal feeding tubes are typically more flexible, softer, and less irritating than the firmer G-tube. The flexible nature and softness of the duodenal feeding tube also improves patient comfort, reducing the incidence of acute sinusitis and other G-tube associated complications. Patients tend to tolerate the duodenal feeding tube for longer periods of time with fewer complaints and far fewer complications directly related to an in-dwelling feeding tube.

However, the structure and design of the duodenal feeding tube typically does not provide for checking residual volumes within the stomach. Although significant gastric residuals are less likely when the tube is properly inserted with the distal end past the pyloric sphincter and into the duodenum, severe and sometimes life-ending complications may occur because of the inability to check for gastric residuals. In addition, tube migration back into the stomach may occur and can go unnoticed until unwanted aspiration occurs. Duodenal feeding tubes typically will collapse, occluding the tube, if aspiration is attempted. Duodenal feeding tubes also typically fail to provide a way to assess the quality of gastric secretions, because the outlets for feeding are in the duodenum, not in the stomach.

### BRIEF SUMMARY OF THE INVENTION

A preferred embodiment of a medical intubation device of this invention is a flexible gastrointestinal aspiration tube assembly or unit having an external end and an insertion or distal end, capable of providing fluids to a patient and aspiration of fluids from the patient simultaneously and/or continuously. The flexible gastrointestinal aspiration tube is used for temporary intubation of the patient. The flexible gastrointestinal aspiration tube of the preferred embodiment comprises three lumens. A first or outer lumen is composed of a relatively soft or pliable first elastomeric material and is adapted for delivery of fluids to the duodenum of the patient. A second or middle lumen is formed interior to or within the outer lumen. The second lumen is made of a more rigid elastomeric material than the first lumen, and allows aspiration of fluids from the stomach of the patient. An optional third or inner lumen is formed interior or within to the second lumen for ventilation of the second lumen. The insertion end of the flexible gastrointestinal aspiration tube is positioned with the duodenum.

The flexible gastrointestinal aspiration tube can be adapted for insertion into the patient via an oral-nasal cavity, such as the nares of the patient. For such embodiments, the first elastomeric material is selected to limit irritation of the oral-nasal cavity when inserted.

In some embodiments, the first, second, and third lumens coterminate at the insertion end of the flexible gastrointestinal aspiration tube. In other embodiments, the second and third lumens terminate between the insertion end and the external end of the tube.

A preferred embodiment may also include a cap member coupled to the insertion end of the tube. The cap member is preferably positioned into one or more of the three lumens at the insertion end of the tube prior to insertion. In some embodiments, the weighted member can have three sections, each adapted for insertion into a separate one of the three lumens. The cap member can be a weighted member.

The medical intubation device may also include a radio-opaque marker or other marker located near the insertion end of the tube, allowing monitoring of the position of the tube.

The first lumen includes a portion that remains external to the patient for delivery of fluids to the patient. One or more openings for discharge of fluids may be formed in the wall of the first lumen proximal to the insertion or distal end of the first lumen for discharge of fluids into the duodenum. The second lumen may also extend external to the patient for connection to an aspiration device, and have one or more insertion end openings through the first lumen for aspiration of fluids from the stomach. The third lumen also extends external to the patient for connection to a ventilation source. The external end of the third lumen may be connected to an external valve to allow insertion of medicines by a syringe.

When a radio-opaque marker is used, the insertion end openings of the second lumen may extend through the marker.

### BRIEF DESCRIPTION OF THE DRAWINGS

FIG. 1 is a perspective view of an entire flexible gastrointestinal tube of a preferred embodiment of the invention;

FIG. 2 is a perspective view of two portions of the tube of FIG. 1;

FIG. 3 is a cross-sectional view of the tube of FIG. 1, showing openings in the wall that forms the first lumen;

FIG. 4 is a cross-sectional view of the tube of FIG. 1, showing openings in the wall which forms the second lumen and openings in the wall forming the third lumen into the second lumen;

FIG. 5 is a perspective view of the external end of the tube of FIG. 1;

FIG. 6 is a cross-sectional cutaway view of the external end of the tube of FIG. 1;

FIG. 7 is a view of the tube of FIG. 1 shown positioned in a patient; and

FIG. 8 is an end view of a cross-section of a weighted member shown in perspective view in FIG. 2.

### DETAILED DESCRIPTION OF THE INVENTION

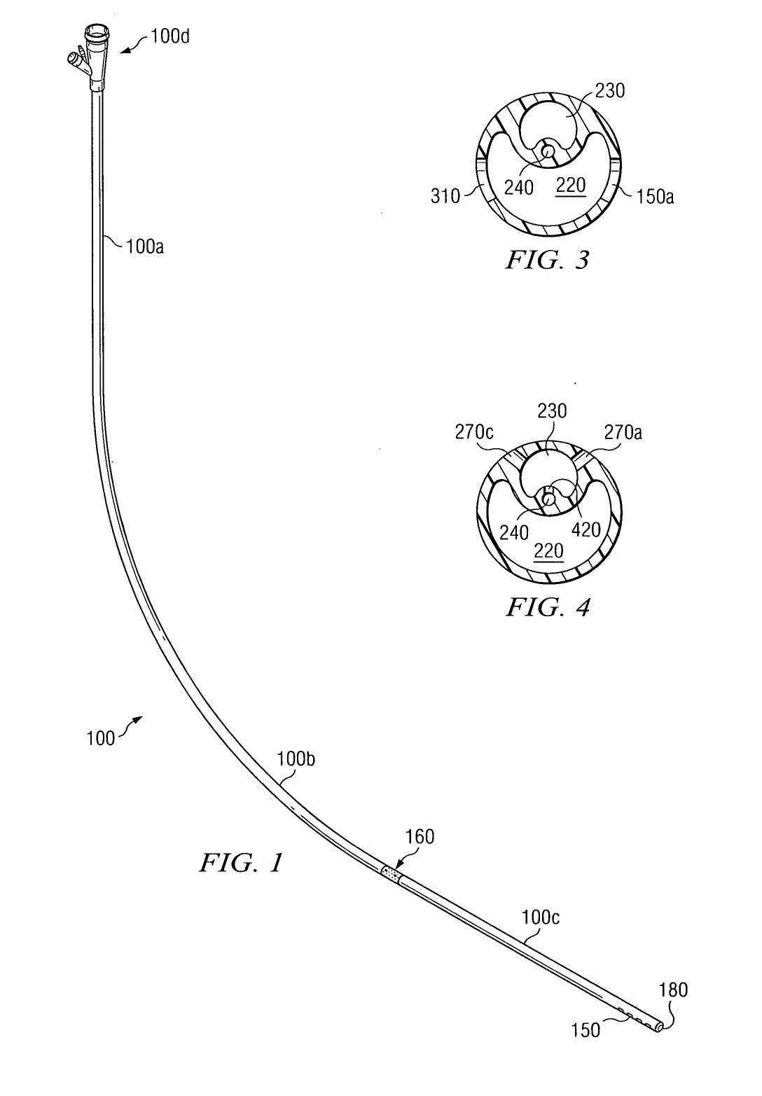

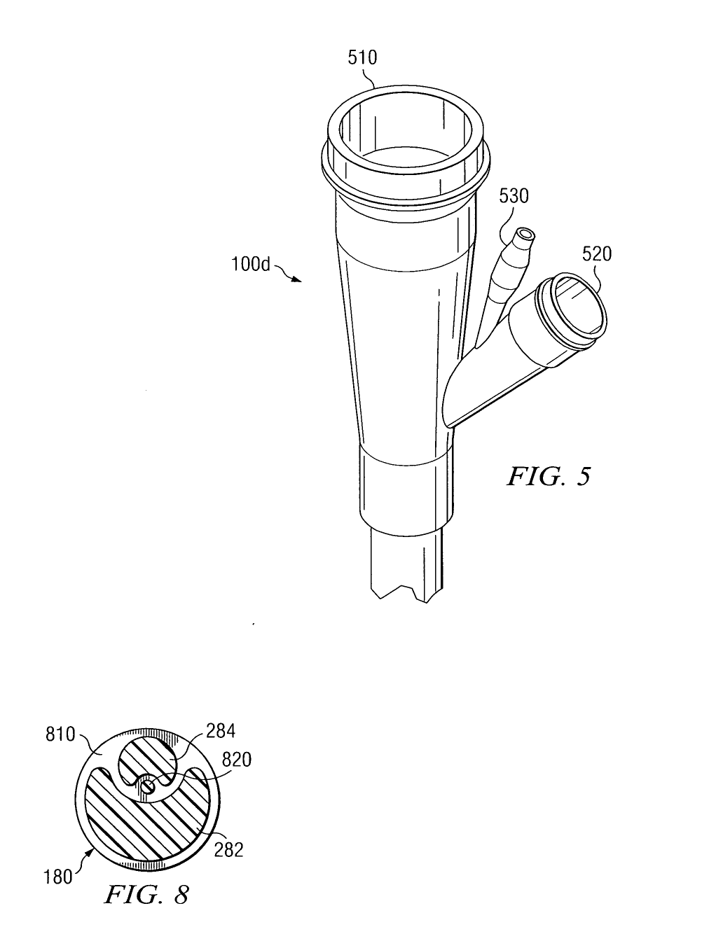

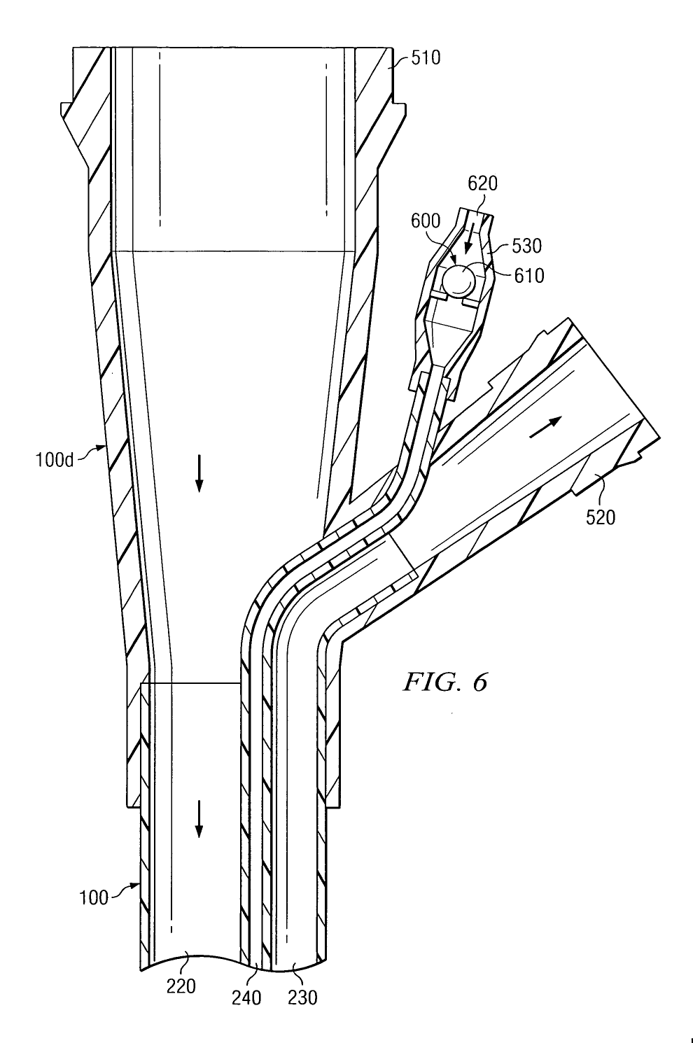

Referring to the drawings, and in particular FIG. 1, the medical intubation device 100 of the preferred embodiment of this invention is illustrated. The intubation device may be described generally as having three sections. A first section 100a is the upper section and includes an adaptor or fitting 100d illustrated in more detail in FIGS. 5 and 6.

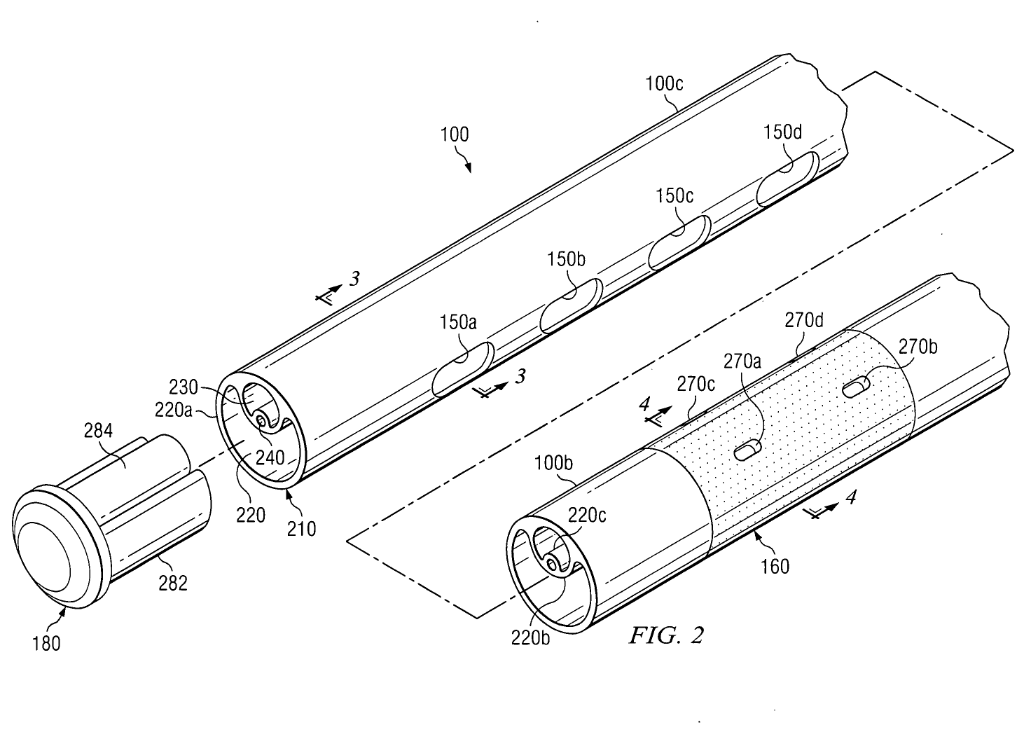

The second section or intermediate section 100b includes a radio-opaque or other marker 160 shown in detail in FIG. 2, and the distal or insertion section 100c includes openings 150 for delivery of sustenance and medicines into the duodenum. The distal section 100c typically terminates at the distal end with a weighted member 180 shown in detail in FIG. 2.

Turning now to FIG. 2, a perspective view shows portions of sections 100b and 100c of a preferred embodiment of the medical intubation tube 100. Section 100c is proximal to an insertion or distal end 210 of the tube 100, illustrating a preferred configuration of a first lumen or passageway 220, a second lumen 230, and a third lumen 240. The third lumen 240 is optional, but preferred. The first or outer lumen 220 is bounded by a generally circular wall 220a of relatively soft first elastomeric material and a second wall portion 220b of a harder or more rigid elastomeric material. This material can be selected from known elastomeric materials to limit irritation of oral-nasal cavity tissue and mucosa when the tube 100 is inserted into a patient and maintained for a long period. The cross-sectional configuration of lumen 220 may be described as generally crescent-shaped, but it should be understood that the internal configuration of lumen 220 may be other suitable shapes functional for the delivery of sustenance and medicines to the duodenum.

Along one side of the interior wall portion 220b of first lumen 220, a second or middle lumen 230 is formed of a second elastomeric material forming a circular wall, which second elastomeric material that is more firm or rigid than the first elastomeric material of wall 220a. The first elastomeric material wall 220a forming the first lumen 220 is designed to be generally collapsible thereby occluding first lumen 220, if aspiration through first lumen 220 was attempted, but the second elastomeric material of the wall portion 220b forming the second lumen 230 can be selected to ensure non-occlusion or substantial collapsing of the second lumen 230 during aspiration. Although typically used for gastric aspiration, the second lumen 230 can also be used for gastric lavage.

As further shown in FIG. 2, the second lumen 230 is formed integral with the interior surface of the wall 220a of the first lumen 220, but may be formed as a separate lumen bonded or otherwise connected to the wall 220a of the first lumen 220 as desired.

The third lumen 240 is formed of a circular wall portion 220c interior to the second lumen 230 for ventilation of the second lumen 230. Ventilation helps prevent gastric mucosal damage, which can occur when suction is applied to the middle lumen 230. The third lumen 240 can be constructed from the second elastomeric material or any other desired elastomeric material. Ventilation, typically ambient air, allows entraining air or other fluids into the third lumen for ventilation, but aspiration of gastric fluids is prevented, as described below.

As shown in FIG. 2, third lumen 240 is positioned approximately medially to the first lumen 220. However, the position of the third lumen 240 within the second lumen 230 is exemplary and illustrative only, and the third lumen 240 can be formed in any interior position within the second lumen 230. Likewise, although shown integral with a wall of the second lumen 230, the third lumen 240 can be formed or made as a separate structure to the wall portion 220b forming second lumen 230, and may be bonded or otherwise attached to the interior surface of the wall 220b of the second lumen 230. Alternately, the third lumen 240 may be formed within the wall 220b of the second lumen 230.

The plurality of openings or apertures 150a-150d are formed in wall 220a and longitudinally spaced along the first lumen 220 in the insertion or distal section 100c. These openings provide fluid communication from the first lumen 220, allowing the feeds to be delivered outwardly of the openings into the duodenum of the patient when the tube 100 is correctly positioned. Although four such openings 150a-150d are shown in FIG. 2, the number, shape, and arrangements of such openings is exemplary and illustrative only, and any number of openings or other arrangement of openings and shapes of openings can be used as desired. For example, additional openings (not visible in FIG. 2) may be formed opposite the openings 150a-150d. Additionally, or alternately, the insertion end 210 of the first lumen 220 can remain open, allowing feeding through the insertion end 210 of the first lumen 220.

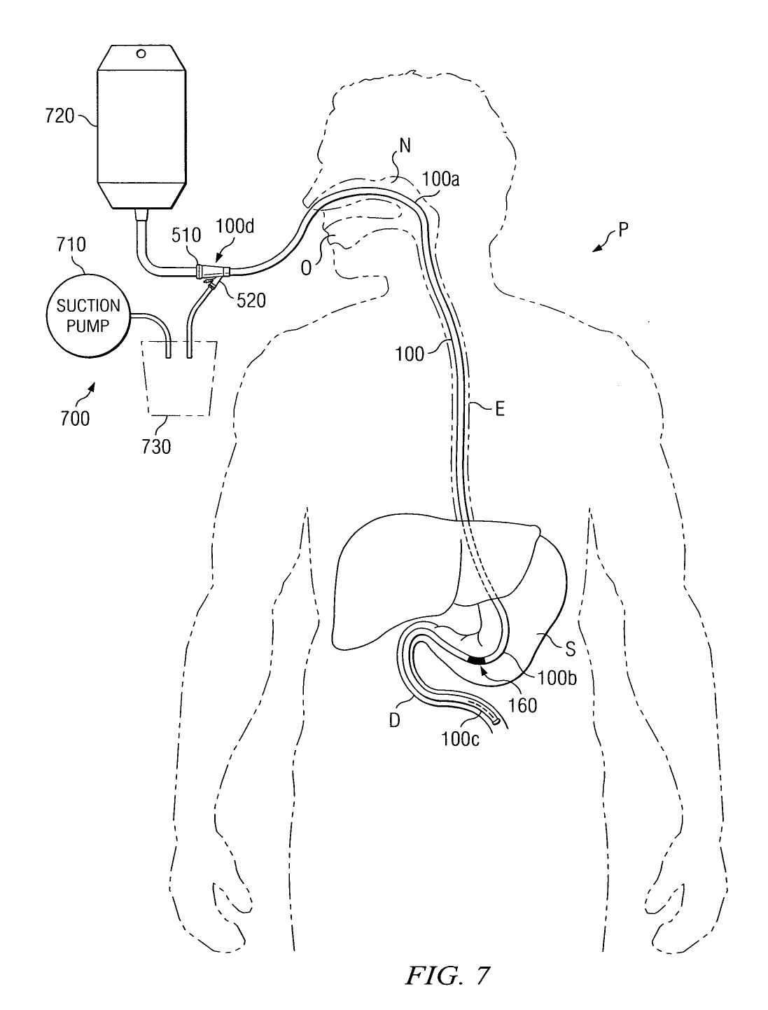

A portion of the second or intermediate section 100b of the tube 100 is also shown in FIG. 2 (the term “intermediate” is not intended literally, but rather denotes the general location). The illustrated portion of section 100b of tube 100 is used for aspiration, and is positioned within the patient's stomach when the tube 100 is fully inserted. In order to monitor the location and placement of the portion 100c, the radio-opaque marker 160 can be placed on or in the tube 100 in section 100b. The marker 160 can be of any suitable material that will be visible to common clinical radiography equipment, and can be applied to the tube 100 in any desired way. For example, the marker 160 can be a sleeve fitted onto the tube 100 or radio-opaque material coated onto the tube 100. Any other desired technique for creating and applying a radio-opaque marker 160 can be used, or another type of locator may be used as is known in the medical arts. The size and shape of the marker 160 is illustrative and exemplary only, and any desired size and shape marker 160 can be used for assisting the positioning of the tube 100 within the stomach of the patient. For example, although the marker 160 is shown in FIG. 7 as being relatively short with respect to the length of the tube 100 in the stomach S, the marker 160 can be made so that it extends substantially the entire length of the stomach S or any other desirable length.

As shown in FIG. 2, in order to allow aspiration, a plurality of openings 270a-270d are formed through the wall portion of wall 220a of first lumen 210, allowing fluid communication between the stomach and the interior of the second lumen 230. The number, arrangement, and shape of openings 270a-270d shown in FIG. 2 are exemplary and illustrative only, and any number, arrangement, and shape of openings 270 can be used. The openings 270 should preferably be within the area marked by the radio-opaque marker 160, to ensure that these openings for aspiration from the stomach can be correctly positioned within the patient's stomach. When second lumen 230 is formed from a separate structure bonded to the interior surface of the outer lumen 220, the second lumen 230 is preferably bonded or otherwise connected to the wall of the outer lumen 220 along the entire length of the second lumen 230, but alternately can be bonded or otherwise connected to the outer lumen wall 220a at selected locations, including proximally to the outlets 270.

To assist the insertion of the tube 100 into the patient and through internal passageways of the patient, a weighted member, such as cap member 180 shown in FIG. 2, can be connected to the insertion or distal end 210 of the tube 100. Any form and method for attaching the cap member 180 can be used that will ensure firm connection of the cap member 180 to the tube 100 proximal to the insertion end 210, preventing dislodgement of the cap member 180 during insertion, use, or removal of the tube 100. The weighted cap member 180 can be constructed of any suitably heavy substance as desirable for assisting the insertion of the tube 100. The use of a weighted cap member 180 is optional, and other techniques for assisting the insertion of the tube 100 can be used. Preferably, a radio-opaque marker (not shown) is provided proximal to the insertion end of the tube 100, for monitoring the positioning of the insertion end of the tube 100. The weighted member may be constructed of materials that provide such a radio-opaque marker. Other techniques for providing a radio-opaque marker can be used, as discussed above.

As shown in FIG. 2, lumens 220, 230 and 240 coterminate at the insertion or distal end of tube 100 and the cap member 180 is formed with separate plug sections 282, 284, and as best shown in FIG. 8, plug section 820, for insertion into lumens 220, 230 and 240, respectively, separately plugging or closing each lumen 220-240. In other embodiments, lumens 230 and 240 terminate distal to the insertion end 210, and a cap member 180 with a single plug section can be used. In yet further embodiments, lumen 220 can be closed by any suitable plug or formation at the distal end 210, with the cap member 180 constructed to attach on an outer surface of the tube 100. Any other desired technique for constructing the cap member 180 and insertion at or in distal end 110 of the lumen 220 can be used. FIG. 8 further shows a cap portion 810 for covering the entire insertion end 210 of the tube 100.

FIGS. 3 and 4 illustrate cross-sections of the tube 100 at locations 3-3 and 4-4, respectively. Cross-sectional view FIG. 3 illustrates an opening 310 positioned in wall portion 220a opposed to opening 150a, providing fluid communication from opposite sides of lumen 220 for delivery of medicines and sustenance into the duodenum. Other similar openings can be placed opposite or generally aligned with openings 150b-150d, if desired. As described above, the position and shape of the one or more openings 310 is exemplary and illustrative only.

Cross-sectional view FIG. 4 illustrates an opening 420 in wall portion 220c, which cooperates with wall portion 220a to form third lumen 240, the opening 420 providing fluid communication between lumen 240 and lumen 230, allowing ventilation of lumen 230 during aspiration or at other desired times, such as a manual flush via lumen 240. Any number of openings and arrangement and shape of openings 420 can be used, as desired. Preferably, the openings 420 are proximal to the openings 270. FIG. 4 also better illustrates the opening 270c, partially obscured in FIG. 2, providing fluid communication between lumen 230 and the patient's stomach. As stated above, the number, shape, and placement of openings 270 is exemplary and illustrative only and any number, shape, and arrangement of openings 270 can be used.

Turning now to FIGS. 5 and 6, fitting or adaptor 100d is provided for attachment to the external section 100a of the medical intubation device. The fitting 100d provides a coupling or flange 510 for connection to a feeding source (not shown), as well as a coupling or flange 520 for connection to an aspiration apparatus (also not shown). The fitting or connector 100d further includes a coupling 530 that allows connection or fluid communication of lumen 240 to a ventilation source, which can include, among others, ambient air and a syringe. In embodiments in which lumen 240 is omitted, fitting 100d can also omit coupling 530. These connections are described in more detail below and in FIG. 7. The tubular portion 530 exits through the wall of coupling 520 as shown in FIG. 5, but this tubular portion can also exit through the wall of coupling 510. The fitting 100d can be constructed from any suitable material, typically a relatively hard plastic material, and can be removably or permanently attached to the tube 100 as desired in order to provide a connection between each of the lumen portions in fitting 100d and the corresponding lumens 220, 230 and 240. Couplings or connectors 510-530 of fitting 100d can be of any desired shape for allowing easy access to each of the three lumens 220-240 and connection of external medical apparatus for fluid communication to lumens 220-240.

FIG. 6 is a sectional view of fitting or adaptor 100d formed by coupling members 510-530, illustrating a preferred way of attaching the fitting and coupling elements to the lumens 220-240. A shown in FIG. 5, the fitting 400 is attached to the exterior wall of the external end of lumen 220, while the wall portions forming lumens 230 and 240 extend beyond the external end of lumen 220 and are directed laterally toward coupling member 520 and 530, through the wall of coupling 510. Lumen 240 extends beyond the external end of lumen 230 through a wall of coupling 520 and terminates at coupling number 530, shown in FIG. 6, which can be formed integral with the rest of fitting 100d if desired. As shown in FIG. 6, coupling 530 provides a ball and cage valve 600, such that valve 600 is open when fluid flows in direction of arrow 620 but closes under positive fluid pressure outward from lumen 240 and the patient's stomach, thereby closing the external end of lumen 240 to fluid passage. Because fluid pressures in the patient's stomach are positive when suction is not being applied to lumen 230, the valve of coupling 530 can prevent leakage of gastric secretions via lumen 240. The valve 600 will remain closed when positive pressures exist in the stomach, and remain open when negative pressures are applied to the lumen 230. The proximal end of the lumen 240 through fitting or connector 510 is designed to be easily accessed with a syringe when manual flush is needed in the directing arrow 620 to relieve obstruction of the lumen 240. Although shown as a ball and cage valve in FIG. 6, valve 600 can be any other desired type of check valve.

Turning now to FIG. 7, a patient P is shown in phantom with the tube 100 inserted through the nares N, down the esophagus E, and into the stomach S and duodenum D. As shown, the tube portion 100c is correctly positioned for duodenal feeding and aspiration in section 100b from the stomach S, as can be checked by using clinical radiography to locate the radio opaque marker or other marker 160, as described above. Both aspiration and feeding can be performed continuously and simultaneously as medically indicated for the patient P. Although shown inserted through the nares N, alternately the tube 100 can be inserted through the oral cavity O, as desired or necessary. A feeding apparatus 720 is connected to coupling 510 of the fitting 100d for providing liquid feeds. As shown, the feeding apparatus is a conventional fluids bag commonly used for enteral feeding. An aspiration apparatus 700 comprises a suction pump 710 connected to a reservoir 730 (shown in phantom), which is in turn connected to coupling 520. The feeding apparatus 720 and aspiration apparatus 700 are exemplary and illustrative only, and other feeding and aspiration apparatus can be used as desired. Although as described above the feeding section 100c is positioned for duodenal feeding, the section 100c can be positioned for jejunal feeding or in other intestinal portions as desired.

The foregoing disclosure and description of the invention are illustrative and explanatory thereof, and various changes in the details of the illustrated apparatus and construction and the method of operation may be made without departing from the spirit of the invention.

1. A medical intubation device, comprising:
a flexible gastrointestinal aspiration generally elongated tubular assembly having an insertion end and an external end for intubation of a patient, comprising:
a first generally longitudinal wall structure forming a first lumen of a first relatively soft elastomeric material to allow some compression to conform to body passages, and adapted for the delivery of fluids to the duodenum or other intestinal area of a patient;
a second generally longitudinal wall structure forming a second lumen formed interiorly to the first lumen of a second elastomeric material, the second elastomeric material being relatively less compressible than the first elastomeric material, adapted for connection to an aspiration device for aspiration of fluids from the stomach of the patient; and
a third generally longitudinal wall structure forming a third lumen, formed interiorly to the second lumen and in fluid communication with the second lumen,
wherein the insertion end of the flexible gastrointestinal aspiration tubular assembly is positioned substantially with the duodenum or other intestinal area of the patient,
whereby delivery of fluids to the duodenum or other intestinal area and aspiration of fluids from the stomach can be performed substantially simultaneously.

2. The medical intubation device of claim 1,
wherein the flexible gastrointestinal tubular assembly is adapted for insertion into the patient via an oral-nasal cavity of the patient, and
wherein the first elastomeric material is selected to limit irritation of the oral-nasal cavity when the flexible gastrointestinal tubular assembly is inserted into the oral-nasal cavity.

3. The medical intubation device of claim 1, wherein the first lumen, second lumen, and third lumen coterminate at the insertion end of the flexible gastrointestinal aspiration tubular assembly.

4. The medical intubation device of claim 1, wherein the first lumen terminates at the insertion end of the flexible gastrointestinal aspiration tubular assembly, and
wherein the second lumen and the third lumen terminate between the insertion and the external end of the flexible gastrointestinal aspiration tubular assembly.

5. The medical intubation device of claim 1, further comprising:
a cap member coupled to the insertion end of the flexible gastrointestinal aspiration tubular assembly.

6. The medical intubation device of claim 5, wherein the cap member is inserted into the insertion end of the flexible gastrointestinal aspiration tubular assembly prior to insertion of the flexible gastrointestinal aspiration tubular assembly into the patient.

7. The medical intubation device of claim 6, wherein the cap member comprises:
a first section, adapted for insertion into the first lumen, closing the first lumen;
a second section, adapted for insertion into the second lumen, closing the second lumen; and
a third section, adapted for insertion into the third lumen, closing the third lumen.

8. The medical intubation device of claim 5, wherein the cap member is a weighted member.

9. The medical intubation device of claim 1, further comprising:
a radio opaque marker coupled to the flexible gastrointestinal aspiration tubular assembly,
whereby the position of the flexible gastrointestinal aspiration tubular assembly can be monitored.

10. The medical intubation device of claim 1, the first lumen comprising:
a first lumen external end, extending external to the patient after positioning of the flexible gastrointestinal aspiration tubular assembly for delivery of fluids to the patient;
a first lumen insertion end, for positioning substantially with the duodenum or other intestinal area of the patient;
an opening for discharge of fluids proximal to the first lumen insertion end.

11. The medical intubation device of claim 1, the second lumen comprising:
a second lumen external end, extending external to the patient after positioning of the flexible gastrointestinal aspiration tubular assembly for connection to an aspiration device;
a second lumen opening providing fluid communication with the stomach of the patient, positioned for aspiration of fluids from the stomach of the patient after positioning of the flexible gastrointestinal aspiration tubular assembly.

12. The medical intubation device of claim 11, further comprising:
a radio opaque marker coupled to the flexible gastrointestinal aspiration tubular assembly proximal to the second lumen opening,
whereby the position of the second lumen opening can be monitored.

13. The medical intubation device of claim 12, wherein the second lumen opening extends through the radio opaque marker.

14. The medical intubation device of claim 11, the third lumen comprising:
a third lumen external end, extending external to the patient after positioning of the flexible gastrointestinal aspiration tubular assembly for ventilation of the second lumen; and
a third lumen opening providing fluid communication between the second lumen and the third lumen proximal to the second lumen opening.

15. The medical intubation device of claim 11, wherein the second lumen opening is positioned within the stomach of the patient when the insertion end of the flexible gastrointestinal aspiration tubular assembly is positioned substantially with the duodenum or other intestinal area.

16. The medical intubation device of claim 1, the third lumen comprising:
a third lumen external end, extending external to the patient after positioning of the flexible gastrointestinal aspiration tubular assembly for ventilation of the second lumen; and
a valve disposed external to the patient coupled to the third lumen external end.

17. The medical intubation device of claim 16, wherein the valve is a check valve.

18. The medical intubation device of claim 16, wherein the valve is adapted for insertion of a syringe.

19. A medical intubation device, comprising:
a flexible gastrointestinal aspiration generally elongated tubular assembly having an insertion end and an external end for intubation of a patient, comprising:
a first generally longitudinal wall structure forming a first lumen of a first relatively soft elastomeric material to allow some compression to conform to body passages, and adapted for the delivery of fluids to the duodenum or other intestinal area of a patient;
a second generally longitudinal wall structure forming a second lumen formed interiorly to the first lumen of a second elastomeric material, the second elastomeric material being relatively less compressible than the first elastomeric material, adapted for connection to an aspiration device for aspiration of fluids from the stomach of the patient,
wherein the insertion end of the flexible gastrointestinal aspiration tubular assembly is positioned substantially with the duodenum or other intestinal area of the patient,
whereby delivery of fluids to the duodenum or other intestinal area and aspiration of fluids from the stomach can be performed substantially simultaneously.

20. The medical intubation device of claim 19, the flexible gastrointestinal aspiration generally elongated tubular assembly further comprising:
a third generally longitudinal wall structure forming a third lumen, formed interiorly to the second lumen and in fluid communication with the second lumen, adapted to provide ventilation to the second lumen.

21. The medical intubation device of claim 19,
wherein the flexible gastrointestinal tubular assembly is adapted for insertion into the patient via an oral-nasal cavity of the patient, and
wherein the first elastomeric material is selected to limit irritation of the oral-nasal cavity when the flexible gastrointestinal tubular assembly is inserted into the oral-nasal cavity.

22. The medical intubation device of claim 19, further comprising:
a radio opaque marker coupled to the flexible gastrointestinal aspiration tubular assembly,
whereby the position of the flexible gastrointestinal aspiration tubular assembly can be monitored.

23. The medical intubation device of claim 19, the first lumen comprising:
a first lumen external end, extending external to the patient after positioning of the flexible gastrointestinal aspiration tubular assembly for delivery of fluids to the patient;
a first lumen insertion end, for positioning substantially with the duodenum or other intestinal area of the patient;
an opening for discharge of fluids proximal to the first lumen insertion end.

24. The medical intubation device of claim 19, the second lumen comprising:
a second lumen external end, extending external to the patient after positioning of the flexible gastrointestinal aspiration tubular assembly for connection to an aspiration device;
a second lumen opening providing fluid communication with the stomach of the patient, positioned for aspiration of fluids from the stomach of the patient after positioning of the flexible gastrointestinal aspiration tubular assembly.

25. The medical intubation device of claim 24, wherein the second lumen opening is positioned within the stomach of the patient when the insertion end of the flexible gastrointestinal aspiration tubular assembly is positioned substantially with the duodenum or other intestinal area.
